(19) United States
(12) Patent Application Publication
(10) Pub. No.: US 2006/0209258 A1
(43) Pub. Date: Sep. 21, 2006

(54) Methods and systems for configuring color documents for colorblind users

(75) Inventors: Ravi K. Nareppa, Rochester, NY (US); Anil K. Menon, Pittsford, NY (US); Bao Vu, Victor, NY (US); Linda S. Bierman, Webster, NY (US); Catherine A. Zehr, Webster, NY (US)

Correspondence Address: OLIFF & BERRIDGE, PLC., P.O. BOX 19928, ALEXANDRIA, VA, 22320, US

(73) Assignee: XEROX CORPORATION, Stamford, CT (US)

(21) Appl. No.: 11/083,034

(22) Filed: Mar. 18, 2005

(51) Int. Cl. A61B 3/02

(52) U.S. Cl. 351/242

(57) ABSTRACT

Methos and systems for configuring a plurality of colors in an image may provides for displaying at least one color test example that corresponds to at least one color vision capability condition, receiving an input selection corresponding to the at least one color vision capability condition, and altering a template for the plurality of colors based on the input selection. The patent or application file contains at least one drawing executed in color. Copies of this patent or patent application publication with color drawings will be provided by the Patent Office upon request and payment of the necessary fee.

### BACKGROUND

This invention relates to configuring color documents for display or print for users with limited color vision.

Human visual acuity typically includes the ability to distinguish across a range of colors in the visible light portion of the electromagnetic spectrum. Some persons are unable to discern between particular sets of colors. Such individuals may thereby lack the capacity to adequately interpret particular color images, whether rendered in hardcopy media or displayed on a monitor, and may be categorized as “colorblind” in particular visual contexts.

A subject's ability to discern between particular colors may be evaluated by presenting images that contain a target within a background. Ishihara plates provide an example of this technique. A collection of filled circles of different sizes and tones are arranged in an apparently random clutter. The target, such as familiar alphanumeric characters, may be rendered in one color, while the background may exhibit a different color. Depending on the type of color deficiency to be evaluated, the subject viewing the Ishihara plates may or may not recognize the target as being distinct from the background. By selecting the Ishihara plate that corresponds to the most significant discernment between the target and the background, the subject's color vision may be categorized.

The Ishihara plates may employ several colors for evaluation using a test pattern. A collection of dots having various randomly distributed sizes and tones may be exhibited to fill a circle. The dots may exhibit a gray level (monochrome) appearance that may lack any discernable brightness pattern, or alternatively a discernable image representing a false target. A pattern conforming to an alphanumeric character or digit may be added by yellow/blue variation to determine yellow/blue discrimination. Another character or digit pattern defined by red/green variation may be added to determine red/green discrimination.

A particular color value may be characterized by hue and tone, and mapped to a particular coordinate in a color coordinate system. Monitor displays may use device dependent additive colors, such as red-green-blue (RGB) color space, while printers may use subtractive colors, such as cyan-magenta-yellow-black (CMYK) color space. Device independent color spaces include XYZ and L*a*b* developed by the Commission Internationale de l'Eclairage (CIE).

### SUMMARY

Various mapping techniques may provide tables and algorithms for transforming from a set of colors distinguishable under normal visual acuity to another set of colors more distinguishable to a person having some form of colorblindness. Existing systems for rendering an image (defined herein as any collection of visually discemable items recorded onto a medium, that may include but are not limited to pictures and text) lack compensation for colorblindness.

Various exemplary embodiments provide methods and systems for configuring a plurality of colors in an image. The method steps performed may include displaying at least one color test example corresponding to a plurality of color vision capability conditions, receiving an input selection corresponding to the plurality of color vision capability condition, and altering a template for the plurality of colors based on the input selection.

Various exemplary embodiments may also include the plurality of test examples being Ishihara plates. In various exemplary embodiments, the plurality of color vision capability conditions may include at least one of normal vision, protanopia, deutanopia, tritanopia and monochrome.

Various exemplary embodiments may include a template for the plurality of colors corresponding to a CMYK color space, for example, for a printer, or a template for the plurality of colors corresponding to an RGB color space, for example, for a monitor screen. Various exemplary embodiments may compensate for luminescence loss for colors at longer wavelength.

### BRIEF DESCRIPTION OF THE DRAWINGS

Various details are described below with reference to the following figures, wherein:

FIG. 1 shows an exemplary flowchart to test and adjust visual rendering for color perception deficiency; and

FIG. 2 shows an exemplary user interface display for a test screen to determine color perception; and

FIG. 3 shows an exemplary block diagram of a system to test and to adjust visual rendering for color perception deficiency.

### DETAILED DESCRIPTION OF EMBODIMENTS

The following detailed description refers to display and print systems. The systems, may refer to, for example, screen monitors and color printers, etc., for sake of clarity and familiarity. However, it should be appreciated that the principles described herein, may be equally applied to any known or later-developed monitors and printers, beyond the examples specifically discussed herein.

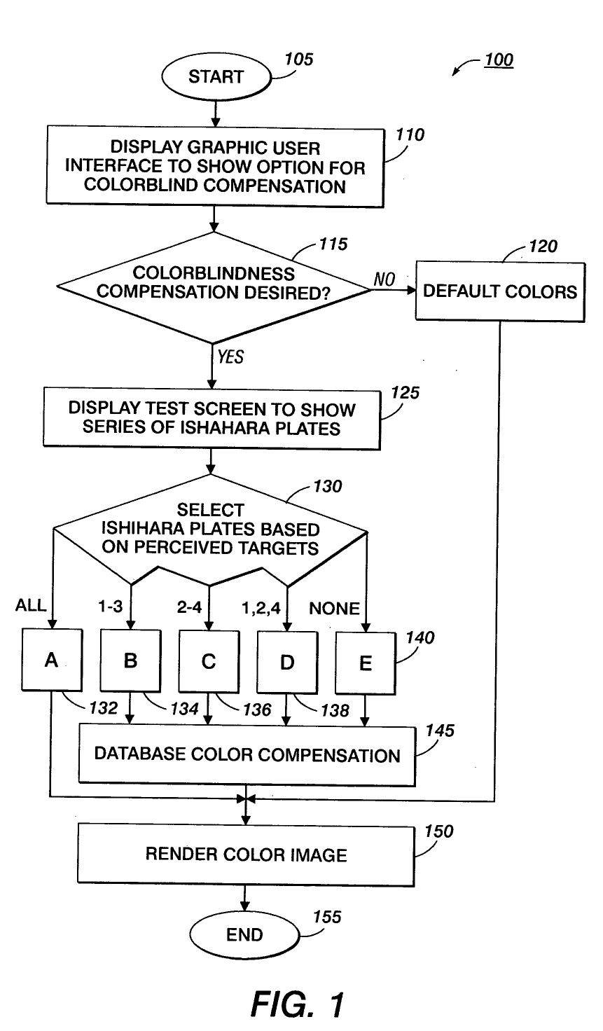

FIG. 1 shows an exemplary flowchart 100 for colorblindness determination and corresponding adjustment in visual display and printing processes. The process begins in step S105 and may proceed to display a graphic user interface to provide an option for colorblindness compensation in step S110. The method may perform a query in step S115. For a negative response, the method branches to step S120 where a preset default color render condition, typically corresponding to normal vision may be input. Otherwise, for a positive response, the method branches to S 125 where a test screen may be displayed, which may feature several Ishihara plates from which to select, described in more detail below.

Based on the options shown in the test screen in step S125, the method may inquire in step S130 which of several Ishihara plates most closely represents the subject's ability to discern the depicted target. The subject (or user) may select all of the plates featuring visible targets for button “A” in step S 132; plates corresponding to protanopia for button “B” in step S134; plates corresponding to deutanopia for button “C” in step S136; plates corresponding to tritanopia for button “D” in step S138; and none of the plates or grayscale monochrome for button “E” in step S140.

Based on the subject's selection, the method may proceed to one of several color rendering conditions, corresponding to the appropriate color compensation database. Upon establishing the corresponding compensation database following one of steps S134 through step S140, the method may continue to step S145 where an appropriate color conversion database to a color compensation process may be applied.

The method then continues to step S150 where an image maybe rendered using colors corresponding to the selected color rendering condition to enable the subject (or user) to visualize the image using the colors best adapted for this purpose. Alternatively or additionally, for either normal selection in step S132 or for the pretest default in step S120, the method skips to step S150 without color compensation to depict the image for the subject. The method then terminates in step S155.

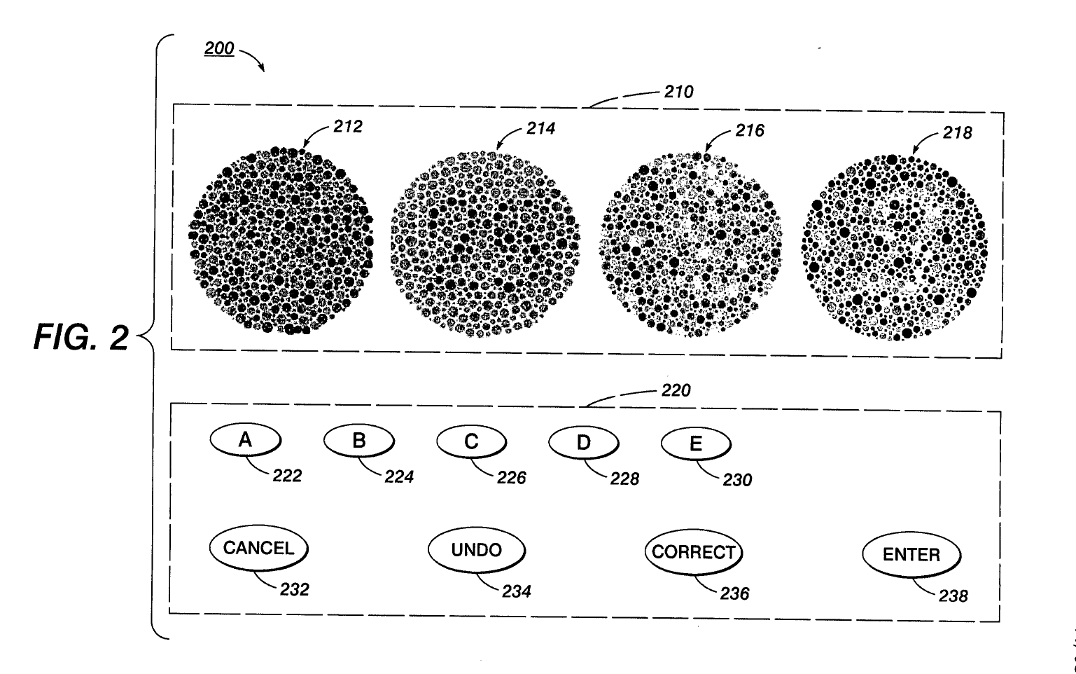

FIG. 2 shows an exemplary user interface display provided by a processor that may provide a test screen 200 to enable self-evaluation of color vision capacity by the user. The screen 200 may display a visual comparison region 210 that may include, for example, four Ishihara plates: a first plate 212 in which all of four colors are visible; a second plate 214 showing the first three of the four colors; a third plate 216 showing the last three of the four colors; and a fourth plate 218 showing all but the second of the colors.

The Ishihara plates may feature one or more alphanumeric characters, such as integer digits. The Ishihara plates, illustrated for the purposes of explanation, each feature a circle having a collection of dots therein, the dots arranged with an apparently random variation of tones and sizes.

The first Ishihara plate 212 displays an orange number “45” as the test object or target among greenish dots, distinguished by differences in hue. The second Ishihara plate 214 displays an orange number “25” as the test object in a comparatively homogeneous green background of dots. The third Ishihara plate 216 displays numbers “6” and “5” as the respective test objects.

Additional patterns may be provided to obscure the target and thereby distract the vision capabilities of the subject being tested. Variations in tone and hue may obscure the target pattern so that a subject with the ability to differentiate between particular colors may correctly perceive the test object, which may be hidden to another subject lacking this visual ability.

The test screen 200 may also display a selection region 220 that may include six buttons, for example. These buttons in region 220 may include a first button 222 labeled “A” for all plates being visible (i.e., the test object being discemable); a second button 224 labeled “B” for the first through third plates being visible, but not the fourth; a third button 226 labeled “C” for the third through fourth plates being visible but not the first; a fourth button 228 labeled “D” for the first, third and fourth plates being visible, but not the second; and a fifth button 230 labeled “E” for none of the plates being visible.

After selecting one of the buttons, the subject may retract the selection by pressing an “UNDO” button 232 or terminate the test by a “CANCEL” button 234. A further correction selection may be selected with correction entry button 236 labeled “CORRECT” to compensate for loss of luminescence experienced for protanopes at the longer wavelengths. Upon deciding the final applicable button corresponding to the subject's perception, the subject may press a selection entry button 238 labeled “ENTER” that may be used to complete the test.

Upon completion of the visual test, a processor for visual display and printing process may automatically apply a transformation mapping from one template to another. The transform may convert color values from those normally perceptible in a standard template to color values in another template based on to the Ishihara plates selected corresponding, to various colorblind templates. The converted color templates may then be used to display the image on the monitor screen and/or printed in hard copy on a substrate sheet.

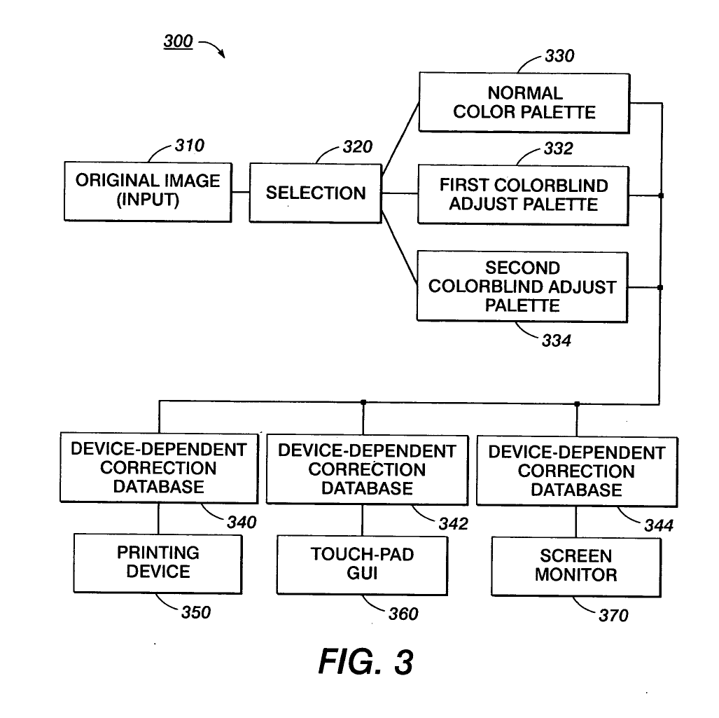

FIG. 3 shows an exemplary color test and compensation system 300. An original image or input 310 may be associated with a selection device 320. Based on results of a color-blindness test from responses by the user (as the subject), a selection device 320 may select one of a normal color palette 330, a first color-blindness adjustment palette 332 and a second color-blindness adjustment 334 palette, as examples.

Adjustment for the first color-blindness criterion may enable access to a first device-dependent correction database 340, a second device-dependent correction database 342 and/or a third device-dependent correction database 344. The first database 340 may correspond to a printing device 350 such as a laser engine, that may produce an output image on a medium. The second database 342 may correspond to a display device such as a touch-pad graphic user interface 360. The third database 344 may correspond to a display device such as a screen monitor 370.

The color test and compensation system 300 may be, in various exemplary embodiments, implemented on a programmed general purpose computer. However, the color test and compensation system 300 may also be implemented on a special purpose computer, a programmed microprocessor or microcontroller in peripheral integrated circuits, an ASIC or other integrated circuit, a digital signal processor, a hard wired electronic or logic circuit such as a discrete element circuit, a programmable logic device such as a PLD, PLA, FPGA or PAL, or the like. In general, any device, capable of implementing a finite state machine that is in turn capable of implementing the flowchart shown in FIG. 1 may be used to implement the color test and compensation system 300.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art which are also intended to be encompassed by the following claims.

What is claimed is:

1. A method of configuring a plurality of colors in an image, comprising:
displaying at least one color test example that corresponds to at least one color vision capability condition;
receiving an input selection that corresponds to one of the at least one color vision capability condition; and
altering a template for the plurality of colors based on the input selection.

2. The method according to claim 1, displaying at least one color test example further comprises:
displaying a plurality of Ishihara plates.

3. The method according to claim 1, wherein the at least one color vision capability condition includes at least one of normal vision, protanopia, deutanopia, tritanopia and monochrome.

4. The method according to claim 1, wherein the template for the plurality of colors corresponds to a CMYK color space.

5. The method according to claim 1, wherein the template for the plurality of colors corresponds to an RGB color space.

6. The method according to claim 1, further comprising:
compensating luminescence loss for longer wavelength colors.

7. A color management system for configuring a plurality of colors in an image, comprising:
a display device that displays at least one color test example corresponding to at least one color vision capability condition;
an input that receives an input selection corresponding to at least one color vision capability condition; and
a processor that alters a template for the plurality of colors based on the input selection.

8. The system according to claim 7, wherein the at least one test example comprises at least one Ishihara plate.

9. The system according to claim 7, wherein the at least one color vision capability condition includes at least one of normal vision, protanopia, deutanopia, tritanopia and monochrome.

10. The system according to claim 7, wherein the template for the plurality of colors corresponds to a CMYK color space.

11. The system according to claim 7, wherein the template for the plurality of colors corresponds to an RGB color space.

12. The system according to claim 7, wherein the process compensates luminescence loss for longer wavelength colors.

13. The system according to claim 7, further comprising a Xerographic device that configures the plurality of colors for the image.

14. A machine-readable storage medium having executable software code for configuring a plurality of colors in an image, the software code comprising:
instructions for displaying at least one color test example corresponding to at least one color vision capability condition;
instructions for receiving an input selection corresponding to the at least one color vision capability condition; and
instructions for altering a template for the plurality of colors based on the input selection.

15. The medium according to claim 14, wherein the at least one test example comprises displaying a plurality of Ishihara plates.

16. The medium according to claim 14, wherein displaying at least one vision capability condition includes at least one of normal vision, protanopia, deutanopia, tritanopia and monochrome.

17. The medium according to claim 14, wherein the template for the plurality of colors corresponds to a CMYK color space.

18. The medium according to claim 14, wherein the template for the plurality of colors corresponds to an RGB color space.

19. The medium according to claim 14, further comprising instructions for compensating luminescence loss for longer wavelength colors.
